(19) United States
(12) Patent Application Publication
(10) Pub. No.: US 2018/0078097 A1
(43) Pub. Date: Mar. 22, 2018

(54) ENCAPSULATED TOOTHPASTE AND DISPENSING DEVICE

(71) Applicant: DANIEL WILLINGHAM, Huston, TX (US)

(72) Inventor: DANIEL WILLINGHAM, Huston, TX (US)

(21) Appl. No.: 15/711,338

(22) Filed: Sep. 21, 2017

Related U.S. Application Data
(60) Provisional application No. 62/397,423, filed on Sep. 21, 2016.

(51) Int. Cl. A47K 5/08; A45D 40/00; B65D 83/04; A61Q 11/00; A61K 8/11

(52) U.S. Cl. CPC A47K 5/08; A45D 40/0075; A61K 8/11; A61Q 11/00; B65D 83/0409

(57) ABSTRACT

An encapsulated toothpaste and dispensing device is disclosed. The encapsulated toothpaste is generally formed into a spherical shape and includes an outer cover portion comprised of a water soluble material adapted to dissolve when in contact with water. The dispensing device adapted to dispense the encapsulated toothpaste onto the head of a toothbrush. The dispenser including a storage portion and dispensing portion. The storage portion adapted to secure and store the encapsulated toothpaste and coupled to the dispensing portion, wherein the encapsulated toothpaste is delivered through a primary aperture within the dispensing portion. The device includes an alignment aid to ensure proper alignment of the toothbrush within the primary aperture.

### CROSS REFERENCE TO RELATED APPLICATIONS

This application claims priority to U.S. Provisional Patent Application No. 62/397,423 filed 21 Sep. 2016 to the above-named inventor, and is herein incorporated by reference in its entirety.

### FEDERALLY SPONSORED RESEARCH OR DEVELOPMENT

Not Applicable

### SEQUENCE LISTING, A TABLE, OR A COMPUTER PROGRAM

Not Applicable

### FIELD OF THE INVENTION

The invention relates generally to an encapsulated toothpaste and dispensing device.

### BACKGROUND

A typical toothpaste dispensing device utilizes a squeezable tube for generally forcing a stream of paste outside of the container and onto a toothbrush of a user. This typical dispensing method and device can be messy and further unnecessarily waste toothpaste as too much toothpaste is placed on the brush head. The dispensing device and method can be especially problematic for children who are just beginning to understand good personal hygiene and brushing habits. Further, it is almost universally accepted that children do not enjoy brushing their teeth and often need encouragement and supervision to ensure that this tooth brushing task is completed.

Within the prior art, to encourage brushing for children, several different and various flavors that are more amenable to children's palates have been utilized. Typically, these flavored pastes are still provided within a tube and can generally be messy.

Accordingly, it would be advantageous to a have a toothpaste that is easier to dispense. Still further, it would be advantageous to have a dispensing device that was specifically adapted to dispense a single serving of toothpaste onto a brush head in a clean manner.

### SUMMARY OF THE INVENTION

The present disclosure provides a toothpaste form adapted for dispensing in a single serving. The toothpaste is generally placed within a water soluble capsule in a spherical shape, wherein individual portions of toothpaste are provided in a single encapsulated sphere. The capsule can be comprised of a water soluble film or other similar material that is dissolved within water and adapted to provide stability for an encapsulated tooth paste. Accordingly, these single servings can be dispensed neatly, cleanly, and efficiently onto a toothbrush head for brushing.

The dispenser of the present disclosure is adapted to dispense the encapsulated sphere portion of toothpaste onto the head of a toothbrush inserted within a primary aperture of the dispenser. The dispenser generally includes a storage portion adapted as a reservoir for the storage of the encapsulated spherical toothpaste portions of the disclosure and a base portion coupled to the storage portion. The base portion generally adapted with the mechanics of the device to release a single encapsulated toothpaste portion from the storage portion and onto the brush head of a toothbrush.

The base portion includes the primary aperture sized and shaped to receive a toothbrush head and position below the storage portion. A sensing means is in communication with the primary aperture and adapted to sense the presence of a toothbrush head inserted into the primary aperture and provide a dispensing signal. Upon receipt of the dispensing signal, a controller directs the release of the encapsulated toothpaste sphere from the storage portion. Accordingly, the encapsulated toothpaste sphere is generally dispensed by placement onto the brush head of the toothbrush within the primary aperture.

Further, to ensure proper alignment of the brush head within the primary aperture, an alignment device can be placed upon the body portion of the toothbrush and adapted with a guide portion sized and shaped for corresponding receipt within a slot adjacent to the primary aperture. Accordingly, the alignment device ensures that the toothbrush head is received within the primary aperture of the base portion in proper alignment with the dispensing mechanism to ensure for placement of the encapsulated toothpaste sphere onto the brush head.

The dispenser and encapsulated toothpaste sphere are generally utilized to make brushing fun for children and provide a cleaner toothpaste dispensing means. Accordingly, the apparatus of the disclosure generally has the like appearance of a gumball dispenser to generally provide familiarity to a child as to the usage of the device of the present invention.

### BRIEF DESCRIPTION OF THE SEVERAL VIEWS OF THE DRAWING(S)

The accompanying drawings are included to provide a further understanding of the present invention and are incorporated in and constitute a part of this specification. The drawings illustrate exemplary embodiments of the present invention and together with the description serve to further explain the principles of the invention. Other aspects of the invention and the advantages of the invention will be better appreciated as they become better understood by reference to the Detailed Description when considered in conjunction with accompanying drawings, and wherein:

FIG. 1 shows a front side view of the dispensing device, according to the present invention;

FIG. 2 shows an isometric view of with the hidden components of the dispensing device, according to the present invention;

FIG. 3 shows an isometric view of the dispensing device, according to the present invention;

FIG. 4 shows a side view with hidden components of the dispensing device according to the present invention;

FIG. 5 shows an isometric view of the alignment device, according to the present invention; and

FIG. 6 shows a side view of the encapsulated toothpaste, according to the present invention.

### DETAILED DESCRIPTION OF THE INVENTION

The following detailed description includes references to the accompanying drawings, which form a part of the detailed description. The drawings show, by way of illustration, specific embodiments in which the invention may be practiced. These embodiments, which are also referred to herein as “examples,” are described in enough detail to enable those skilled in the art to practice the invention. The embodiments may be combined, other embodiments may be utilized, or structural, and logical changes may be made without departing from the scope of the present invention. The following detailed description is, therefore, not to be taken in a limiting sense.

Before the present invention is described in such detail, however, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the disclosure made herein.

Unless otherwise indicated, the words and phrases presented in this document have their ordinary meanings to one of skill in the art, Such ordinary meanings can be obtained by reference to their use in the art and by reference to general and scientific dictionaries.

References in the specification to “one embodiment” indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The following explanations of certain terms are meant to be illustrative rather than exhaustive. These terms have their ordinary meanings given by usage in the art and in addition include the following explanations.

As used herein, the term “and/or” refers to any one of the items, any combination of the items, or all of the items with which this term is associated.

As used herein, the singular forms “a,” “an,” and “the” include plural reference unless the context clearly dictates otherwise.

As used herein, the terms “include,” “for example,” “such as,” and the like are used illustratively and are not intended to limit the present invention.

As used herein, the terms “preferred” and “preferably” refer embodiments of the invention that may afford certain benefits, under certain circumstances. However, other embodiments may also be preferred, under the same or other circumstances.

Furthermore, the recitation of one or more preferred embodiments does not imply that other embodiments are not useful, and is not intended to exclude other embodiments from the scope of the invention.

As used herein, the term “coupled” means the joining of two members directly or indirectly to one another. Such joining may be stationary in nature or movable in nature and/or such joining may allow for the flow of fluids, electricity, electrical signals, or other types of signals or communication between two members. Such joining may be achieved with the two members or the two members and any additional intermediate members being integrally formed as a single unitary body with one another or with the two members or the two members and any additional intermediate members being attached to one another. Such joining may be permanent in nature or alternatively may be removable or releasable in nature.

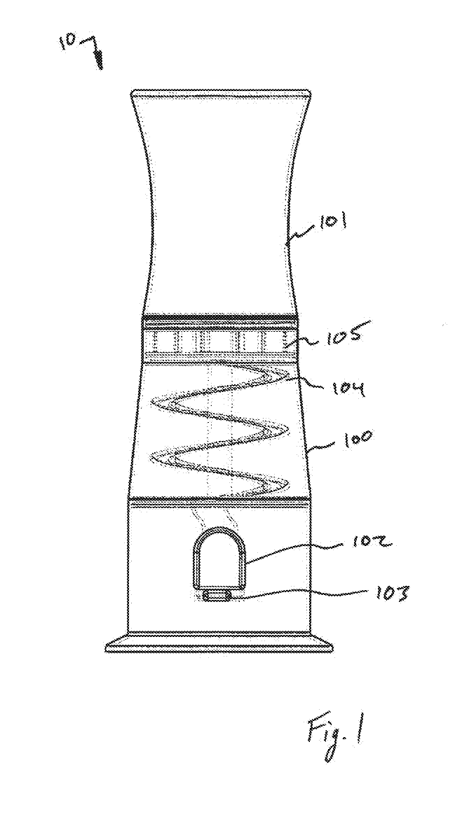

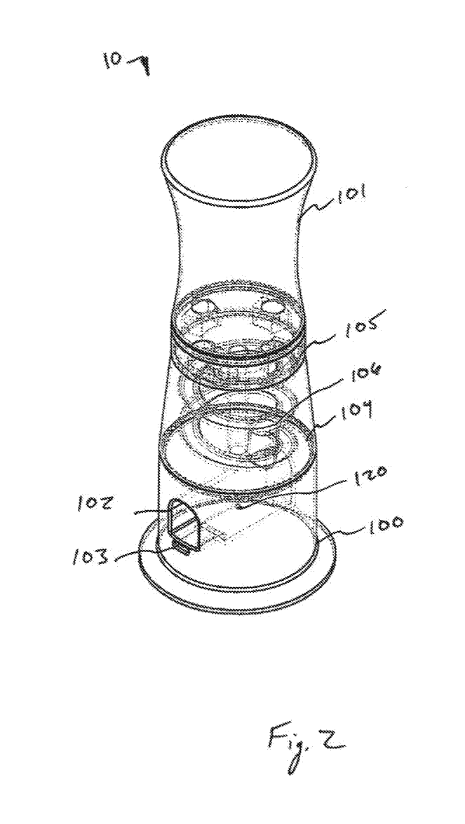

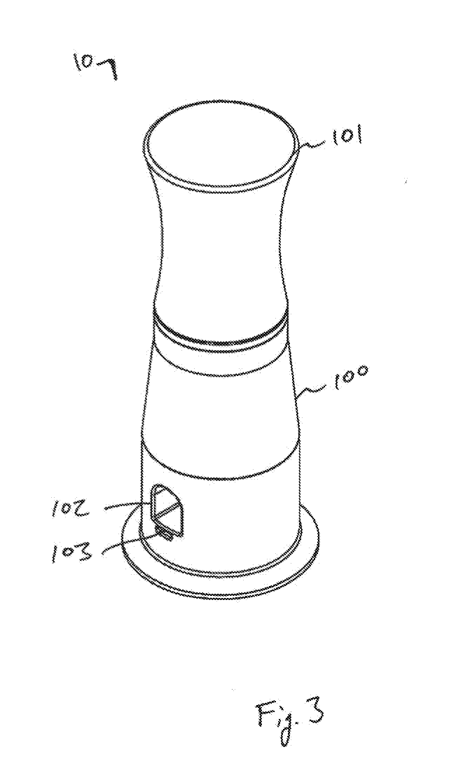

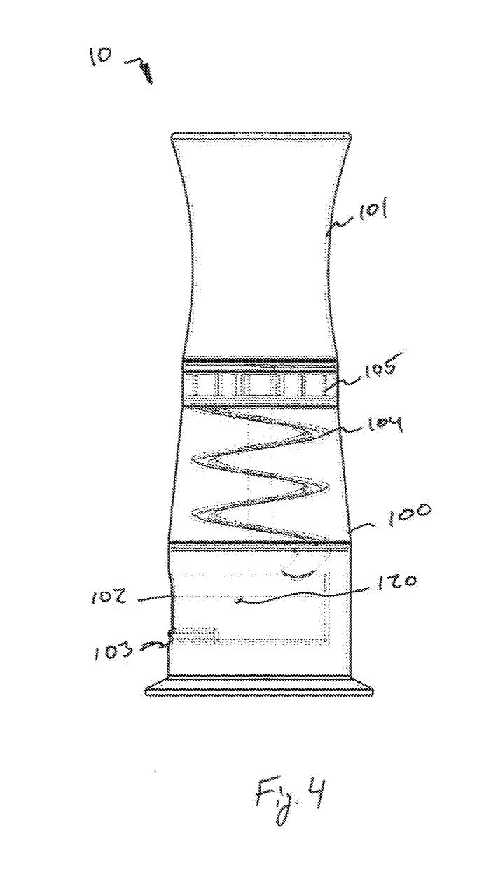

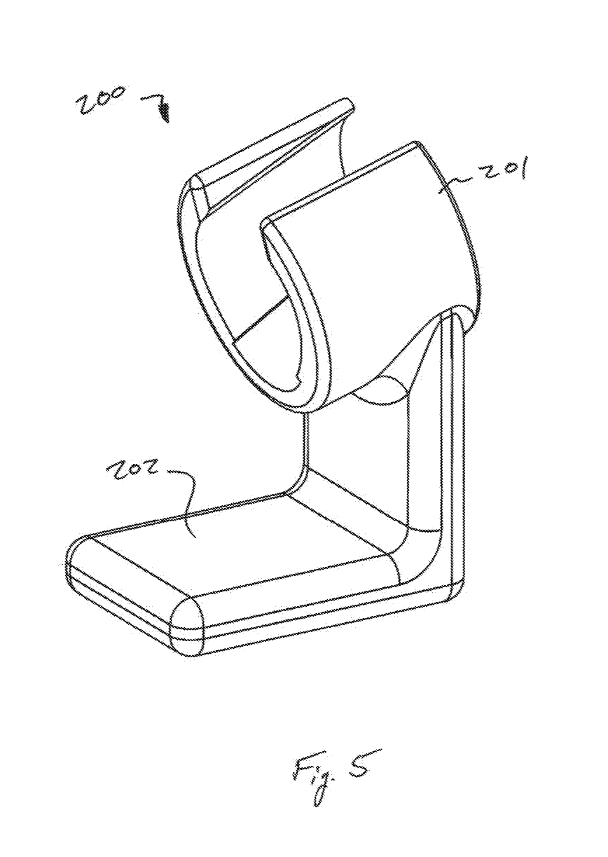

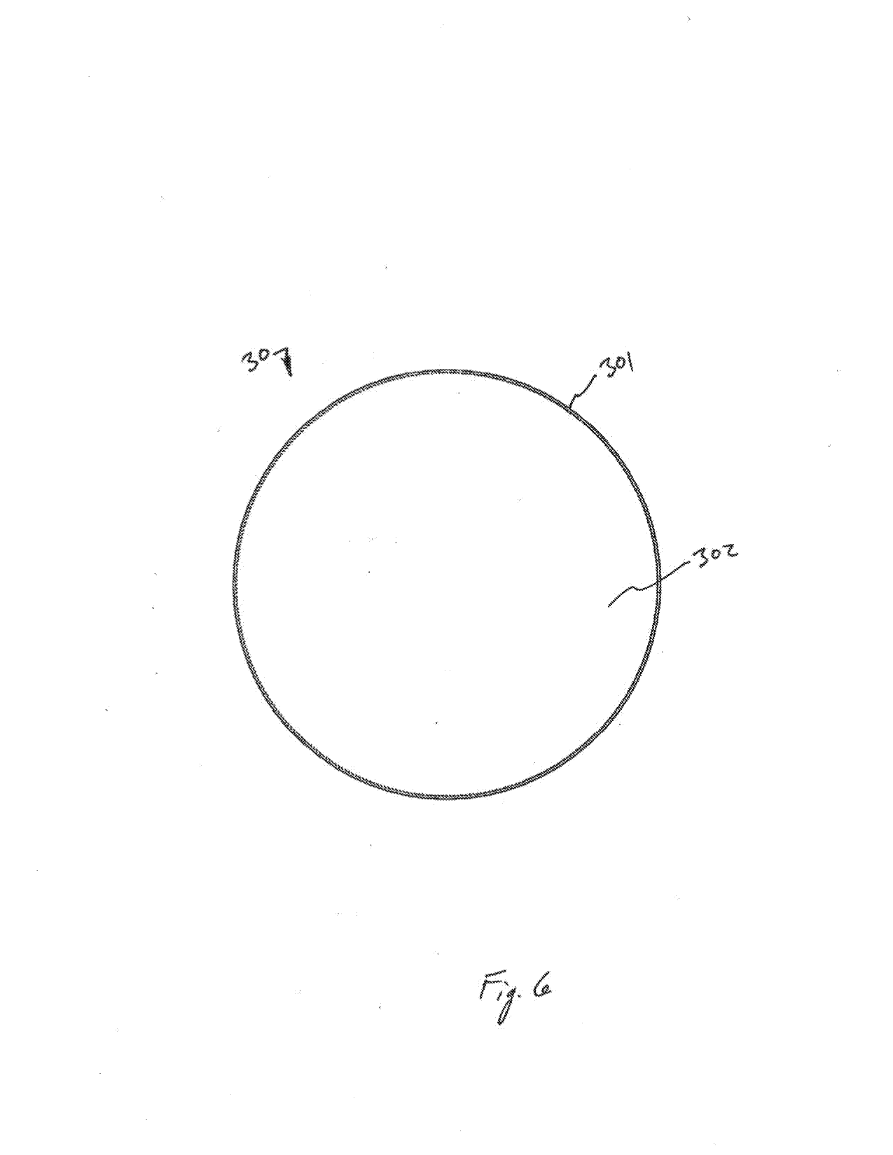

Referring now to FIG. 1-FIG. 6 an encapsulated toothpaste 30 and dispenser 10 adapted to dispense the encapsulated toothpaste 30 is shown. The encapsulated toothpaste 30 of the present disclosure is generally formulated from a paste adapted for cleaning teeth (“toothpaste”) 302 encapsulated within a water soluble outer cover 301. The outer cover 301 is selected from a material that is dissolved upon contact with water wherein the outer cover 301 may be, but not limited to, a film, a capsule, a membrane, or other similar water soluble material generally providing an enclosure to encapsulate a paste 302. The encapsulated toothpaste 30 is preferably provided in a spherical shape and wherein the encapsulated toothpaste 30 resembles a ball.

The dispenser 10 of the present disclosure is generally adapted to dispense the encapsulated toothpaste 30 onto a toothbrush of a user. The dispenser 10 is provided in an assembly including a base portion 100 and a storage portion 101 removably received on the base portion 100. The storage portion 101 providing an enclosure for storage of the encapsulated toothpaste 30 prior to dispensing by the device 10. The storage portion 101 extending a height opposite the base portion 100 and preferably constructed of a translucent material, wherein the quantity of encapsulated toothpaste 30 portions is easily seen.

The base portion 100 adapted to dispense the encapsulated toothpaste 30 portions upon demand and direction by the user of the device 10. Accordingly, the base portion 100 includes mechanical components and electrical components to effectuate this dispensing.

The base portion 100 having a primary aperture 102 providing access to an interior of the base portion 100 and aligned with a dispensing mechanism 105 of the device 10, wherein the dispensing mechanism 105 is adapted to allow for the release of a single encapsulated toothpaste 30 from the storage portion 101 and into the primary aperture 102. Once received in the primary aperture 102, the encapsulated toothpaste 30 can be accessed by a user of the device 10 and placed upon a toothbrush for brushing.

In the preferred embodiment of the present disclosure, the device 10 base portion 100 includes a slot 103 placed adjacent to the primary aperture 102 and adapted to receive a guide portion 202 of an alignment device 200 received on a toothbrush, wherein the alignment device 200 ensures proper orientation of a toothbrush within the primary aperture 102 for the placement of the encapsulated toothpaste 30 onto the brush portion of the toothbrush.

The alignment device 200 is adapted for receipt on a neck of a toothbrush adjacent to the brush head of the toothbrush. Accordingly, the alignment device 200 includes a clip portion 201, the clip portion 201 adapted to provide grasping support to the neck and secure the alignment device 200 to the toothbrush. The guide portion 202 extending from the clip portion 201 parallel to the brush head and sized and shaped for receipt into the slot 103, wherein placement of the guide portion 202 into the slot 103 properly positions the brush head in alignment with the dispensing mechanism 105.

Preferably, the base portion 100 includes various electronic elements including a power source adapted to provide power to the various features of the device 10. Accordingly, the base portion 100 includes a controller 106 coupled to a sensor 120 positioned within an interior of the primary aperture 102. The sensor 120 adapted to send a signal upon detection of a brush head within the primary aperture 102, wherein the controller 106 relays the signal of the sensor 120 to the dispensing mechanism 105 for dispensing of the encapsulated toothpaste.

In the preferred embodiment of the present disclosure, the device 10 includes a track portion 104 coupled to and extending from the dispensing portion 105 to the primary aperture 102. This track portion 104 generally guiding a spherical encapsulated toothpaste 30 portion to the primary aperture 102 through gravity, wherein the track 104 may include an arcuate shape or sidewalk to ensure that the encapsulated toothpaste 30 remains on the track 104 until delivery to the primary aperture 102. Preferably, this track portion 104 is helical in shape and visible to a user of the device 10. Accordingly, the base portion 100 may be provided with designated regions constructed of a generally transparent material, wherein the track portion 104 and travel of the encapsulated toothpaste 30 is visible during use of the device 10.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) but that the invention will include all embodiments falling with the scope of the appended claims.

1. A material for placement within the oral cavity of a user for generally cleaning the teeth of a user, the material comprising:
an outer cover portion, the outer cover portion comprised of a water soluble material, wherein the outer cover is adapted to dissolve upon contact with water; and
a paste, the paste encapsulated within the outer cover portion, the paste formulated to clean the teeth of the user, wherein the paste is accessible upon dissolution of the outer cover.

2. The material as in claim 1, wherein the material is formed into a sphere forming an encapsulated toothpaste.

3. The material as in claim 2, wherein the outer cover is comprised of a film layer.

4. The material as in claim 2, wherein the outer cover is comprised of a capsule.

5. The material as in claim 2, wherein the outer cover is comprised of a membrane.

6. A dispenser adapted to dispense the material of claim 2, the dispenser comprising:
a storage portion, the storage portion providing an enclosure for a plurality of the encapsulated toothpaste spheres, the storage portion removably received on a base portion, and
the base portion including:
a dispensing mechanism, the dispensing mechanism in communication with the storage portion, the dispensing mechanism adapted to release at least one of the encapsulated toothpaste spheres from the storage portion; and
a primary aperture, the primary aperture in communication with the dispensing mechanism, the primary aperture sized and shaped to receive a brush head of a toothbrush, wherein insertion of the brush head into the primary aperture activates the dispensing mechanism for the deposit of the encapsulated toothpaste sphere onto the brush head.

7. The dispenser as in claim 6, wherein the base portion includes a slot, the slot positioned adjacent the primary aperture, the slot sized and shaped to receive an alignment aid, the alignment aid coupled to the toothbrush, the alignment aid adapted to align the toothbrush head with the dispensing mechanism within the primary aperture.

8. The dispenser as in claim 6, wherein the base portion includes a controller, the controller coupled to a sensor and the dispensing mechanism, the sensor generating a signal upon placement of a brush head into the primary aperture, the controller receiving the signal and directing the dispensing of an encapsulated toothpaste sphere from the dispensing mechanism.

9. The dispenser as in claim 6, wherein the base portion includes a track portion, the track portion positioned between and coupled to the dispensing mechanism and the primary aperture, wherein the encapsulated toothpaste sphere travels along the track portion to the primary aperture during dispensing.

10. The dispenser as in claim 9, wherein the track portion is helical in shape.

11. A dispenser adapted to dispense the material of claim 2, the dispenser comprising:
a storage portion, the storage portion providing an enclosure for a plurality of the encapsulated toothpaste spheres, the storage portion removably received on a base portion, and
the base portion including:
a dispensing mechanism, the dispensing mechanism in communication with the storage portion, the dispensing mechanism adapted to release at least one of the encapsulated toothpaste spheres from the storage portion;
a primary aperture, the primary aperture in communication with the dispensing mechanism, the primary aperture sized and shaped to receive a brush head of a toothbrush, wherein insertion of the brush head into the primary aperture activates the dispensing mechanism for the deposit of the encapsulated toothpaste sphere onto the brush head;
a sensor, the sensor positioned within the primary aperture and generating a signal upon insertion of the brush into the primary aperture; and
a controller, the controller coupled to the dispensing mechanism and the sensor, the controller activating the dispensing mechanism upon receipt of the signal.

12. The dispenser as in claim 11, wherein the base portion includes a slot, the slot positioned adjacent the primary aperture, the slot sized and shaped to receive an alignment aid, the alignment aid coupled to the toothbrush, the alignment aid adapted to align the toothbrush head with the dispensing mechanism within the primary aperture.

13. The dispenser as in claim 11, wherein the base portion includes a track portion, the track portion positioned between and coupled to the dispensing mechanism and the primary aperture, wherein the encapsulated toothpaste sphere travels along the track portion to the primary aperture during dispensing.

14. The dispenser as in claim 13, wherein the track portion is helical in shape.

15. A dispenser adapted to dispense at least one encapsulated toothpaste sphere, the dispenser comprising:
a storage portion, the storage portion providing an enclosure for the at least one encapsulated toothpaste sphere, the storage portion removably received on a base portion, and
the base portion including:
a dispensing mechanism, the dispensing mechanism in communication with the storage portion, the dispensing mechanism adapted to release the at least one encapsulated toothpaste sphere from the storage portion;
a primary aperture, the primary aperture in communication with the dispensing mechanism, the primary aperture sized and shaped to receive a brush head of a toothbrush, wherein insertion of the brush head into the primary aperture activates the dispensing mechanism for the deposit of the at least one encapsulated toothpaste sphere onto the brush head;
a sensor, the sensor positioned within the primary aperture and generating a signal upon insertion of the brush into the primary aperture; and
a controller, the controller coupled to the dispensing mechanism and the sensor, the controller activating the dispensing mechanism upon receipt of the signal.

16. The dispenser as in claim 15, wherein the base portion includes a slot, the slot positioned adjacent the primary aperture, the slot sized and shaped to receive an alignment aid, the alignment aid coupled to the toothbrush, the alignment aid adapted to align the toothbrush head with the dispensing mechanism within the primary aperture.

17. The dispenser as in claim 15, wherein the base portion includes a track portion, the track portion positioned between and coupled to the dispensing mechanism and the primary aperture, wherein the encapsulated toothpaste sphere travels along the track portion to the primary aperture during dispensing.

18. The dispenser as in claim 17, wherein the track portion is helical in shape.
